(19) United States
(12) Patent Application Publication
(10) Pub. No.: US 2026/0257052 A1
(43) Pub. Date: Sep. 3, 2026

(54) A MULTI-LAYER DISINFECTION DEVICE FOR DISINFECTING A FEMALE MEDICAL CONNECTOR

(71) Applicant: Becton, Dickinson and Company, Franklin Lakes, NJ (US)

(72) Inventors: Manjunath Mangumali, Bangalore (IN); Deepak Udupa, Mangalore (IN); Ganapathy P., Madurai (IN); Sumit Raosaheb Mane, Ahmednagar, Maharashtra (IN)

(73) Assignee: Becton, Dickinson and Company, Franklin Lakes, NJ (US)

(21) Appl. No.: 19/129,440

(22) PCT Filed: Nov. 30, 2022
(86) PCT No.: PCT/US22/80691

§ 371 Date: May 13, 2025

(51) Int. Cl. A61M 39/16; A61L 2/18; A61L 2/235; A61L 101/34; A61L 101/44; A61L 103/15; A61M 39/10

(52) U.S. Cl. CPC A61M 39/162; A61L 2/18; A61L 2/235; A61L 2101/34; A61L 2101/44; A61L 2103/15; A61L 2202/123; A61M 2039/1044

(57) ABSTRACT

A disinfection device for disinfecting a female medical connector. The device includes an inner housing having a flange and first internal threads that engage first external threads of the medical connector. The device also includes an outer housing having second internal threads that engage second external threads of the inner housing. The outer housing also includes an absorbent material having a disinfectant or antimicrobial agent. Rotation of the outer housing provides frictional engagement between the absorbent material and the medical connector and moves the outer housing toward the inner housing. The second internal and second external threads are configured to provide sufficient scrubbing time and scrubbing rotations by the absorbent material to disinfect the medical connector. The outer housing further includes at least one resilient snap element that moves to an extended position after contact with the flange to indicate that disinfection of the medical connector is complete.

### TECHNICAL FIELD

The present disclosure generally relates to a device for disinfecting and sterilizing female medical connectors, and more particularly, to a disinfection device that includes inner and outer housings that are threadably engaged wherein second external threads of the inner housing and second internal threads of the outer housing are configured to provide sufficient scrubbing time and scrubbing rotations by absorbent material to disinfect the medical connector wherein the outer housing includes at least one resilient snap element to provide an indication that disinfection of the medical connector is complete when the snap element moves to an extended position after being deflected.

### BACKGROUND

Vascular access devices (VAD's) are commonly used therapeutic devices and include intravenous (IV) catheters. There are two general classifications of VAD's, peripheral catheters and central venous catheters. Bacteria and other microorganisms may gain entry into a patient's vascular system from access hubs, ports or valves upon connection to the VAD when delivering a fluid or pharmaceutical. Each access hub, port, valve or connection is associated with some risk of transmitting a catheter related bloodstream infection (CRBSI), which can be costly and potentially lethal.

In order to decrease CRBSI cases and to ensure VAD's are used and maintained correctly, standards of practice have been developed, which include disinfecting and cleaning procedures. Disinfection caps have been added to the Society for Healthcare Epidemiology of America (SHEA) guidelines and caps are also incorporated into the Infusion Nurses Standards (INS) guidelines.

In developed markets, when utilizing an IV catheter, a needleless connector will typically be used to close off the system and then subsequently accessed to administer medication or other necessary fluids via the catheter to the patient. INS Standards of Practice recommend the use of a needleless connector and state that it should be “consistently and thoroughly disinfected using alcohol, tincture of iodine or chlorhexidine gluconate/alcohol combination prior to each access.” The disinfection of the needleless connector is ultimately intended to aid in the reduction of bacteria that could be living on the surface and possibly lead to a variety of catheter related complications including CRBSI. Nurses will typically utilize a 70% isopropyl alcohol (IPA) pad to complete this disinfection task by doing what is known as “scrubbing the hub.” However, compliance to this practice is typically very low. In addition to a lack of compliance to “scrubbing the hub”, it has also been noted through clinician interviews that there is often a variation in scrub time, dry time and the number of times the needleless connector is scrubbed.

Throughout the sequence of procedures associated with the transmission of a microorganism that can cause a CRBSI, there are many risks of contact or contamination. Contamination can occur during drug mixing, attachment of a cannula, and insertion into the access hub. Because the procedure to connect to a VAD is so common and simple, the risk associated with entry into a patient's vascular system has often been overlooked. Presently, the risk to hospitals and patients is a substantial function of the diligence of the clinician performing the connection, and this diligence is largely uncontrollable. In particular, it has been found that health care providers may not completely understand the impact of not following scrubbing protocols. As a result, a health care provider may not disinfect the hub for the recommended amount of time.

Thus, there is a need for a disinfection cap that enables disinfection of the needleless connector while also enhancing compliance to the clinical practice of “scrubbing-the-hub” before accessing the line.

### SUMMARY

An aspect of the present disclosure pertains to a disinfection device for disinfecting a female medical connector having first external threads and an access surface. According to an exemplary embodiment of the present disclosure, the device generally comprises an inner housing, first internal threads, second external threads, a flange, an outer housing, an absorbent material that includes a disinfectant or antimicrobial agent, second internal threads and at least one resilient snap element. The inner housing includes an inner wall that defines a hole that extends through the inner housing. The first internal threads are formed on the inner wall and threadably engage the first external threads in order to attach the medical connector. The second external threads are formed on a housing outer surface of the inner housing and the flange is formed on a flange end of the inner housing. The outer housing includes a chamber defined by a chamber wall, wherein the chamber forms a chamber open end having an end surface. The absorbent material extends through the chamber and hole and contacts the access surface. The second internal threads are formed on the chamber wall and engage the second external threads to enable rotation of the outer housing and absorbent material to provide frictional engagement between the absorbent material and the access surface. In accordance with the present disclosure, the second internal and second external threads are configured to provide sufficient scrubbing time and scrubbing rotations by the absorbent material to disinfect the access surface. During rotation of the housing, the outer housing moves toward the inner housing. The snap element is located on the end surface, wherein contact between the snap element and flange during movement of the outer housing toward the inner housing causes the snap element to deflect from an extended position. The snap element returns to the extended position when the flange clears the snap element wherein positioning of the snap element in the extended position after being deflected indicates that disinfection of the access surface is complete.

In one or more embodiments, a thread pitch of the second external threads is selected to provide sufficient scrubbing time and scrubbing rotations to actively disinfect the access surface.

In an embodiment, the second external threads are configured as fine threads as defined by the Unified Thread Standard.

In an embodiment, the second external threads include a breakable fin element that stops rotation of the inner housing relative to the outer housing to facilitate initial thread engagement between the first internal threads and first external threads when attaching the female connector to the inner housing.

In one or more embodiments, the snap element provides audible and visual indication that disinfection is complete.

In one or more embodiments, the snap element contacts the flange to hold the inner housing within the outer housing to enable removal of the female medical connector from the disinfection device by disengaging the first external threads of the female medical connector from the first internal threads of the inner housing.

In one or more embodiments, the absorbent material is attached to a mounting element extending from a closed end of the outer housing.

In one or more embodiments, the female medical connector is selected from a group consisting essentially of needleless connectors, catheter luer connectors, stopcocks, or hemodialysis connectors.

In an embodiment, the female medical connector is a needleless connector having an access surface, septum, hub surface and first external threads.

In one or more embodiments, the disinfectant or antimicrobial agent is selected from a group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorhexidine, chlorhexidine diacetate, chlorhexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof.

In one or more embodiments, the disinfectant or antimicrobial agent comprises at least one of chlorhexidine gluconate and chlorhexidine diacetate.

In one or more embodiments, the disinfectant or antimicrobial agent is a fluid or a gel.

In another exemplary embodiment of the present disclosure, the device generally comprises an inner housing, first internal threads, second external threads, a flange, an outer housing, a mounting element, an absorbent material that includes a disinfectant or antimicrobial agent, second internal threads and at least one resilient snap element. The inner housing includes an inner wall that defines a hole that extends through the inner housing. The first internal threads are formed on the inner wall and threadably engage the first external threads. The second external threads are formed on a housing outer surface of the inner housing and the flange is formed on a flange end of the inner housing and wherein the flange includes a flange outer surface. The outer housing includes a closed end and a chamber defined by a chamber wall, wherein the chamber forms a chamber open end having an end surface. The mounting element extends from the closed end of the outer housing. The absorbent material is attached to the mounting element, wherein the absorbent material extends through the chamber and hole. The second internal threads are formed on the chamber wall and engage the second external threads to enable rotation of the outer housing and absorbent material to provide frictional engagement between the absorbent material and the access surface. In accordance with the present disclosure, the second internal and second external threads are configured to provide sufficient scrubbing time and scrubbing rotations by the absorbent material to actively disinfect the access surface. During rotation of the outer housing, the outer housing moves toward the inner housing to compress the absorbent material and release the disinfectant or antimicrobial agent such that the disinfectant or antimicrobial agent contacts the hub portion to passively disinfect the hub portion. The snap element is located on the end surface, wherein contact between the snap element and flange during movement of the outer housing toward the inner housing causes the snap element to deflect from an extended position. The snap element returns to the extended position when the flange clears the snap element wherein positioning of the snap element in the extended position after being deflected indicates that disinfection of the access surface is complete. In the extended position, the snap element also contacts the flange outer surface to hold the inner housing within the outer housing.

In a further exemplary embodiment of the present disclosure, the device generally comprises an inner housing, first internal threads, second external threads, a flange including a flange outer surface, an outer housing, inner gear teeth, a mounting element, an absorbent material that includes a disinfectant or antimicrobial agent and second internal threads. The inner housing includes an inner wall that defines a hole that extends through the inner housing. The first internal threads are formed on the inner wall and threadably engage the first external threads. The second external threads are formed on a housing outer surface of the inner housing. The flange is formed on a flange end of the inner housing wherein the flange outer surface includes a plurality of outer gear teeth each having a first slope and a second slope steeper than the first slope. The outer housing includes a closed end and a chamber defined by a chamber wall, wherein the chamber forms a chamber open end having an end surface. The inner gear teeth are located at the chamber open end and extend from the chamber wall. Each inner tooth includes first and second slopes that correspond in profile to the first and second slopes of the outer teeth. The mounting element extends from the closed end of the outer housing. The absorbent material is attached to the mounting element, wherein the absorbent material extends through the chamber and hole. The second internal threads are formed on the chamber wall and engage the second external threads to enable rotation of the outer housing and absorbent material to provide frictional engagement between the absorbent material and the access surface. The second internal and second external threads are configured to provide sufficient scrubbing time and scrubbing rotations by the absorbent material to actively disinfect the access surface. During rotation of the outer housing, the outer housing moves toward the inner housing to compress the absorbent material and release the disinfectant or antimicrobial agent such that the disinfectant or antimicrobial agent contacts the hub portion to passively disinfect the hub portion. Upon disinfection of the access surface, the inner teeth engage the outer teeth to cause a click sound due to movement between respective slopes of the inner and outer teeth wherein the click sound provides audible indication that disinfection of the access surface is complete.

### BRIEF DESCRIPTION OF THE DRAWINGS

FIG. 1 is a cross sectional view of a multi-layer device for disinfecting a female connector according to an embodiment of the present disclosure.

FIG. 2 is a perspective view of an inner housing of the device.

FIG. 3 is a perspective view of assembled inner and outer housings.

FIG. 4A is a view of the device aligned with a female connector on a center axis.

FIG. 4B depicts a hub portion of a needleless connector inserted in a second end of the inner housing.

FIG. 4C depicts at least one snap element in an extended position wherein the snap element engages a flange outer surface after movement of the outer housing toward the inner housing.

FIG. 4D is a view of the needleless connector and device after the needleless connector is removed from the device.

FIGS. 5A and 5B show perspective and end views, respectively, of an alternate embodiment of a multi-layer device for disinfecting a needleless connector.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

Embodiments of the present disclosure pertain to a multi-layer disinfection device for disinfecting a female medical connector. The device includes inner and outer housings that are threadably engaged. In particular, the inner housing includes a flange and first internal threads that engage first external threads of the medical connector. The device also includes an outer housing having second internal threads formed on a housing outer surface that engage second external threads of the inner housing. The outer housing also includes an absorbent material having a disinfectant or antimicrobial agent. Rotation of the outer housing provides frictional engagement between the absorbent material and the medical connector and moves the outer housing toward the inner housing. The second internal and second external threads are configured to provide sufficient scrubbing time and scrubbing rotations by the absorbent material to disinfect the medical connector. In one or more embodiments, the outer housing further includes at least one resilient snap element that moves to an extended position after contact with the flange to indicate that disinfection of the medical connector is complete.

With respect to terms used in this disclosure, the following definitions are provided.

As used herein, the use of “a,” “an,” and “the” includes the singular and plural.

As used herein, the term “catheter related bloodstream infection” or “CRBSI” refers to any infection resulting from the presence of a catheter or IV line.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is generally associated with a flush syringe and can interlock and connect to the female end located on the vascular access device (VAD). A Luer connector comprises a distal end, a proximal end, an irregularly shaped outer wall, a profiled center passageway for fluid communication from the chamber of the barrel of a syringe to the hub of a VAD. A Luer connector also has a distal end channel that releasably attaches the Luer connector to the hub of a VAD, and a proximal end channel that releasably attaches the Luer connector to the barrel of a syringe.

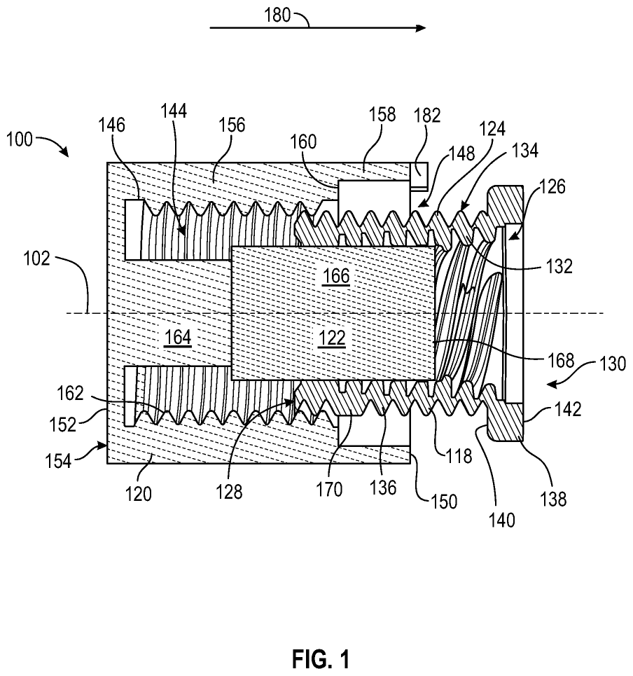

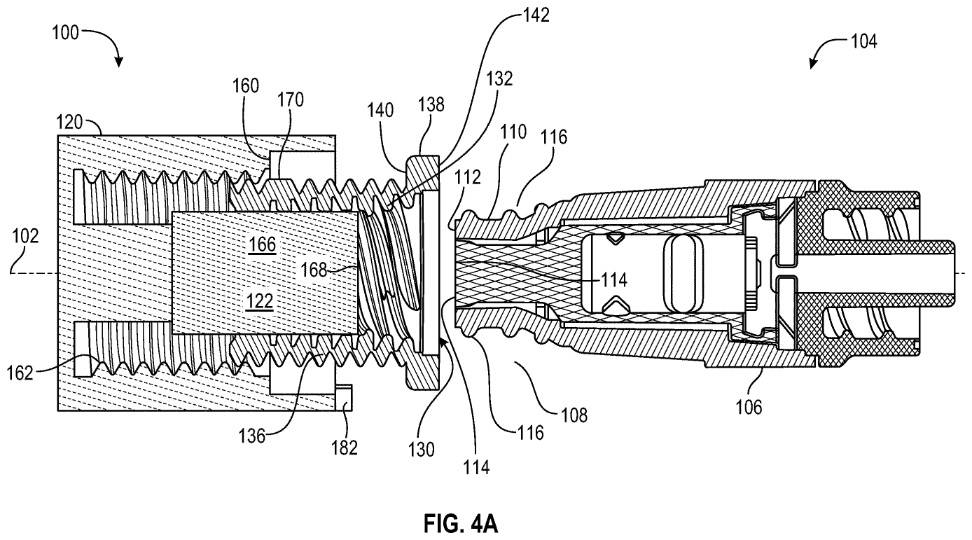

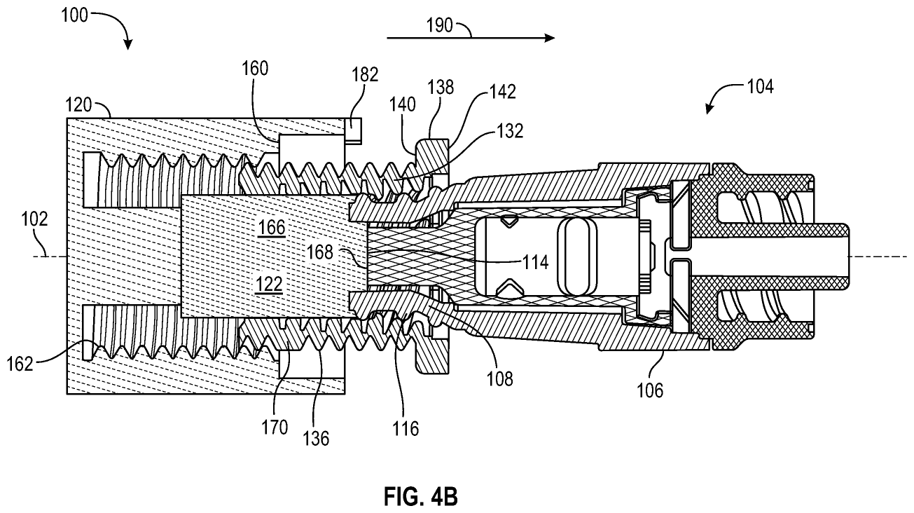

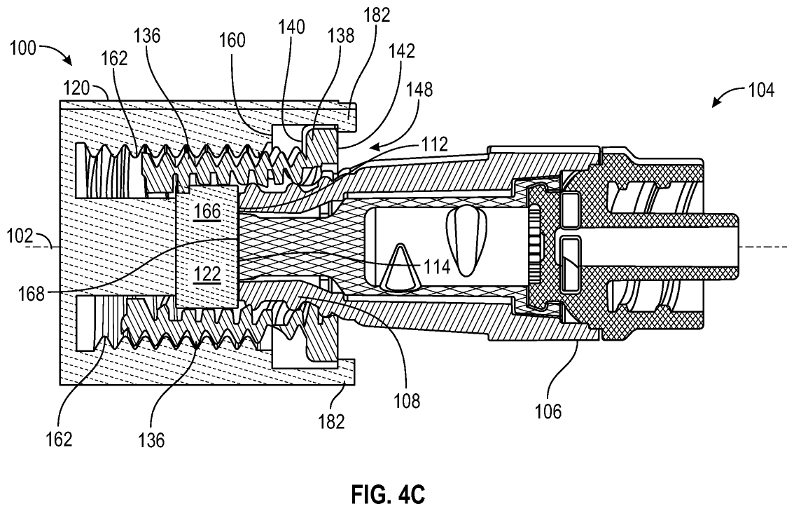

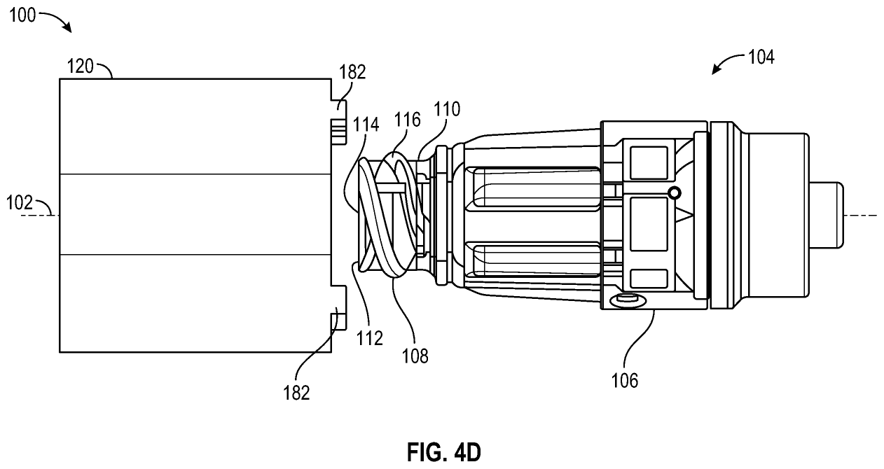

Referring to FIG. 1, a cross sectional view of a multi-layer device 100 for disinfecting a female connector is shown arranged relative to a center axis 102 of the device 100. In accordance with the present disclosure, a female connector 104 (see FIG. 4A) may comprise a needleless connector, catheter luer connector, stopcock, or a hemodialysis connector. For purposes of illustration, the present disclosure will be described when used in conjunction with a needleless connector 106. In one or more embodiments, the needleless connector 106 includes a hub portion 108 having a substantially cylindrically shaped outer hub surface 110 oriented substantially parallel to the center axis 102. The hub portion 108 further includes an access surface 112 that includes a septum 114. The access surface 112 and septum 114 are oriented substantially transverse to the hub surface 110. The hub surface 110 further includes first external threads 116. In an embodiment, the first external threads 116 comply with screw thread standards such as that set forth by the International Organization for Standardization (ISO).

The device includes an inner cap or housing 118, an outer cap or housing 120 and absorbent material 122. The inner housing 118 includes an inner housing wall 124 that defines a hole 126 that extends between first 128 and second 130 ends of the inner housing 118. The inner housing wall 124 includes first internal threads 132 having a size and pitch configured to engage the first external threads 116 such as threads that comply with ISO standard threads.

In one or more embodiments, a housing outer surface 134 of the inner housing 118 includes second external threads 136 having a thread pitch that is selected to provide sufficient scrubbing time and scrubbing action or scrubbing rotations to actively disinfect an access surface of a female connector 104, such as access surface 112 of needleless connector 106. In an embodiment, the second external threads 136 of the inner housing 118 are configured as single start fine threads that comply with a screw thread standard such as that set forth in the Unified Thread Standard (UTS). The inner housing 118 further includes a flange 138 located at the second end 130 having flange inner 140 and outer 142 surfaces.

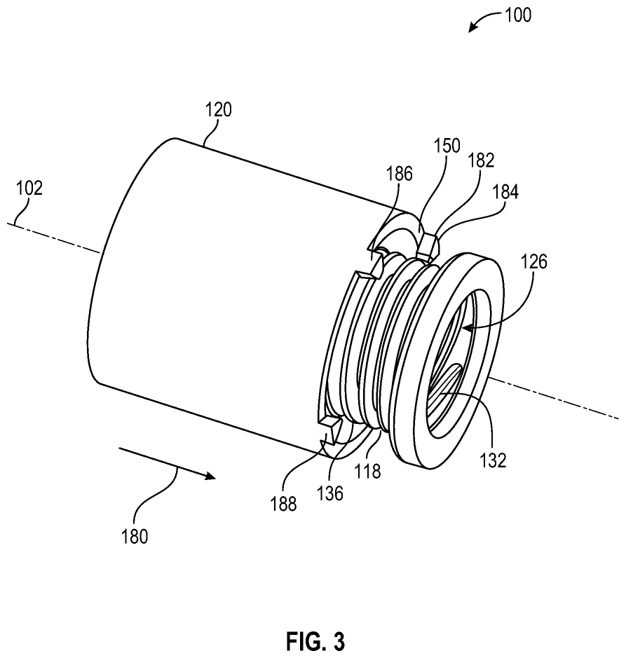

The outer housing 120 includes a chamber 144 defined by a chamber wall 146. The chamber 144 extends through a first end of the outer housing 120 to form a chamber open end 148 having an annular end surface 150 (see FIG. 3). The outer housing 120 also includes an end wall 152 located at a second end 154 of the outer housing 120 that closes the chamber 144. The chamber wall 146 includes a thread wall portion 156, flange wall portion 158 located at the chamber open end 148 and a transverse shoulder wall portion 160 located between the thread 156 and flange 158 wall portions. The thread wall portion 156 of the outer housing 120 includes second internal threads 162 having a size and pitch or thread profile configured to engage the second external threads 136 of the inner housing 118 such as threads that comply with UTS standard threads. The inner housing 18 is received by the chamber open end 148 in order to threadably engage the second external threads 136 and second internal threads 162 to enable rotation of the outer housing 120 relative to the inner housing 118. A diameter of the flange wall portion 158 is sized to receive the flange 138 thus reducing a thickness of the flange wall portion 158 relative to the thread wall portion 156.

The outer housing 120 further includes a mounting element 164 that extends from the end wall 152 and into the chamber 144. The absorbent material 122 is attached to an end of the mounting element 164 such that the absorbent material 122 extends through the chamber 144, first end 128 and into the hole 126 of the inner housing 118. The absorbent material 122 includes a disinfectant or an antimicrobial agent 166 for disinfecting a female connector 104. In one or more embodiments, the absorbent material 122 may be a nonwoven material, foam, or a sponge having a porous structure. In a specific embodiment, the foam is a polyurethane foam. In a specific embodiment, the absorbent material 122 is a sponge. The absorbent material 122 may be attached to the mounting element 164 by an adhesive or other attachment method.

Absorbent material 122 retains disinfectant or antimicrobial agent 166 in its porous structure due to surface tension and releases disinfectant or antimicrobial agent 166 when squeezed or compressed by an incoming female connector 104. The device 100 is designed to be compatible in interacting with various disinfectants. In one or more embodiments, the disinfectant or antimicrobial agent 166 may include variations of alcohol or chlorhexidine. In one or more embodiments, the disinfectant or antimicrobial agent 166 is selected from the group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorhexidine, chlorhexidine diacetate, chlorhexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof. In a specific embodiment, the disinfectant or antimicrobial agent 166 comprises at least one of chlorhexidine gluconate and chlorhexidine diacetate. In one or more embodiments, the disinfectant or antimicrobial agent 166 is a fluid or a gel. In one or more specific embodiments, the disinfectant or antimicrobial agent 166 is 70% isopropyl alcohol (IPA).

The absorbent material 122 includes an engagement surface 168 that contacts the access surface 112 of the needleless connector 106. Rotation of the outer housing 120 causes corresponding rotation of the absorbent material 122 and engagement surface 168 to provide frictional engagement between the engagement surface 168 and the access surface 112. This provides a scrubbing action on the access surface 112 to actively disinfect the access surface 112.

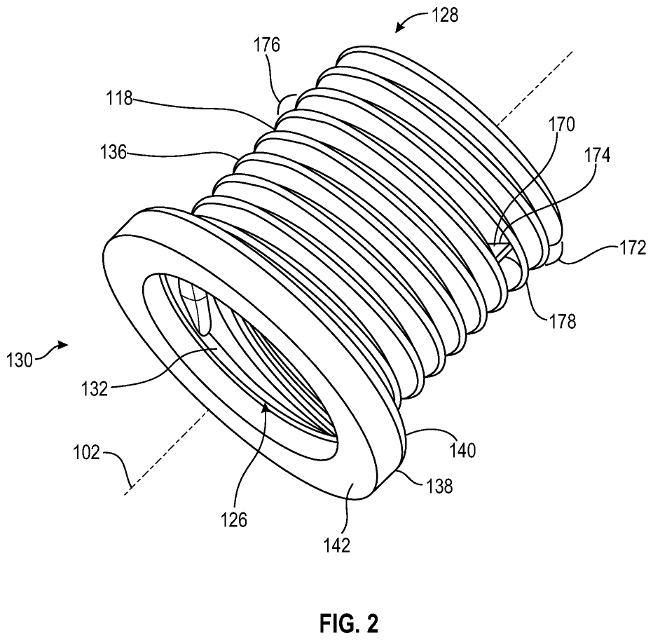

Referring to FIG. 2, a perspective view of the inner housing 118 is shown. Referring to FIG. 2 in conjunction with FIG. 1, the second external threads 136 include a breakable fin element 170 and a first thread section 172 located at the first end 128 of the inner housing 118. In one or more embodiments, the fin element 170 may be an upstanding element 174 that is oriented substantially transverse to an orientation of the second external threads 136. The fin element 170 is located after the first thread section 172 and between a pair of adjacent threads 176. The fin element 170 serves to block engagement of the second external threads 136 of the inner housing 118 and second internal threads 162 of the outer housing 120 after the first thread section 172. This stops rotation of the inner housing 118 relative to the outer housing 120 which in turn facilitates initial thread engagement between the first internal threads 132 of the inner housing 118 and first external threads 116 when attaching the needleless connector 106 to the inner housing 118. In an embodiment, the fin element 170 has a substantially rectangular shape and includes an edge 178 oriented substantially transverse to the second external threads 136.

The fin element 170 is configured to break upon the application of sufficient torque or twisting force on the outer housing 120 in order to continue engagement of the second internal threads 162 and second external threads 136 after initial thread engagement. Referring back to FIG. 1, the outer housing 120 is rotated and moves toward the inner housing 118 (see arrow 180) along the center axis 102. When this occurs, the flange 138 is received by the flange wall portion 158.

Referring to FIG. 3, a perspective view of assembled inner 118 and outer 120 housings is shown. Referring to FIG. 3 in conjunction with FIG. 1, the outer housing 120 further includes at least one snap element 182 that extends from the end surface 150 toward the center axis 102. In one or more embodiments, the outer housing 120 includes first 184, second 186 and third 188 snap elements. FIG. 1 depicts the snap element 182 in an extended position wherein the snap element 182 is positioned to interfere or contact a portion of the flange 138 as the outer housing 120 is rotated and moves toward the inner housing 118. In an embodiment, the snap element 182 and outer housing 120 are fabricated from a resilient material. Contact between the snap element 182 and the flange 138 as the outer housing 120 moves toward the inner housing 118 causes the snap element 182 to deflect and move to a deflected position, thus providing clearance for the flange 138. The snap element 182 returns, or snaps back, to the extended position when the flange 138 moves past, or clears, the snap element 182 to indicate that disinfection of the access surface 112 is complete. Upon return of the snap element 182 to the extended position, the snap element 182 contacts the flange outer surface 142 thus securing the flange 138.

FIGS. 4A-4D depict operation of the device in accordance with the present disclosure. In FIG. 4A, the device 100 is shown aligned with a female connector 104 on center axis 102. As previously described, the female connector 104 may be a needleless connector, catheter luer connector, stopcock, or hemodialysis connector. For purposes of illustration, the present disclosure will now be described when used in conjunction with a needleless connector 106 having an access surface 112 that includes a septum 114. The access surface 112 and septum 114 are oriented substantially transverse to a hub surface 110. The hub surface 110 further includes first external threads 116 for threadably engaging the needleless connector 106 with a known male connector of a syringe or other medical device or medical implement.

Referring to FIG. 4B, the hub portion 108 of needleless connector 106 is shown inserted in the second end 130 of the device 100. In order to attached the needleless connector 106 to the device 100, the device 100 is moved toward the needleless connector 106 (see arrow 190) such that the hub portion 108 is received by the second end 130. The outer housing 120 is then rotated to engage the first internal threads 132 with the first external threads 116. As previously described, the fin element 170 stops rotation of the inner housing 118 relative to the outer housing 120 which in turn facilitates initial thread engagement between the first internal threads 132 and first external threads 116. Upon full engagement of the first internal threads 132 and first external threads 116, the engagement surface 168 contacts the access surface 112.

A torque or twisting force is then applied to the outer housing 120 sufficient to break the fin element 170. This enables continued rotation of the outer housing 120 in order to continue engagement of the second internal threads 162 and second external threads 136. Rotation of the outer housing 120 causes corresponding rotation of the absorbent material 122 and thus engagement surface 168 to provide frictional engagement between the engagement surface 168 and the access surface 112. This provides a scrubbing action on the access surface 112 for a sufficient amount of time that actively disinfects the access surface 112 (see FIG. 4C). In one or more embodiments, the second internal threads 162 and second external threads 136 have a thread pitch selected to provide sufficient scrubbing time and scrubbing rotations by the absorbent material 122 to actively disinfect the access surface 112. In an embodiment, the second external threads 136 are configured as single start fine threads that comply with the Unified Thread Standard (UTS) although it is understood that other thread types may be used. In one or more embodiments, the amount of thread pitch may be increased or decreased based on the time and number of scrubbing rotations by the absorbent material 122 needed to disinfect hub portion 108. The second internal threads 162 and second external threads 136 are configured such that rotation of the outer housing 120 stops upon disinfection of the access surface 112.

Rotation of the outer housing 120 also moves the outer housing 120 toward the inner housing 118 along the center axis 102. This compresses the absorbent material 122 (see FIG. 4C) and releases the disinfectant or antimicrobial agent 166 from the absorbent material 122 such that the disinfectant or antimicrobial agent 130 contacts the access surface 112, septum 114, hub surface 110 and first external threads 116 to provide passive disinfection of access surface 112, septum 114, hub surface 110 and first external threads 116.

The flange 138 is received by the flange wall portion 158 as the outer housing 120 moves toward the inner housing 118. The snap element 182 is positioned to interfere or contact a portion of the flange 138 as the outer housing 120 is rotated and moves toward the inner housing 118. In an embodiment, the snap element 182 and outer housing 120 are fabricated from a resilient material. Contact between the snap element 182 and a portion of the flange 138, such as the flange inner surface 140, causes the snap element 182 to deflect and move to a deflected position, thus providing clearance for the flange 138 and enabling further movement of the outer housing 120 toward the inner housing 118. In one or more embodiments, the access surface 112 is disinfected when the access surface 112 is scrubbed for a sufficient amount of time and undergoes a sufficient number of scrubbing rotations or scrubbing actions by the absorbent material 122. When disinfection of the access surface 112 is complete, the rotation of the outer housing 120 stops and the snap element 182 moves past, or clears, the flange 138. The snap element 182 then returns, or snaps back, to the extended position and engages the flange outer surface 142 when the snap element 182 moves past the flange 138 to indicate that disinfection of the access surface 112 is complete. FIG. 4C depicts the snap element 182 in the extended position wherein the snap element 182 engages the flange outer surface 142 after movement of the outer housing 120 toward the inner housing 118.

Engagement of the snap element 182 with the flange outer surface 142 of the flange 138 provides positive visual and audible indication to a user such as a clinician, nurse or other health care provider that disinfection is complete. Further, engagement of the snap element 182 provides indication that the device 100 has already been used so as to avoid re-use of the device 100. Engagement of the snap element 182 with the flange outer surface 142 also retains or holds the inner housing 118 within the outer housing 120. This enables removal of the needleless connector 106 from the device 100 by disengaging the first external threads 116 of the needleless connector 106 from the first internal threads 132 of the inner housing 118. Referring to FIG. 4D, the needleless connector 106 is shown removed from the device 100.

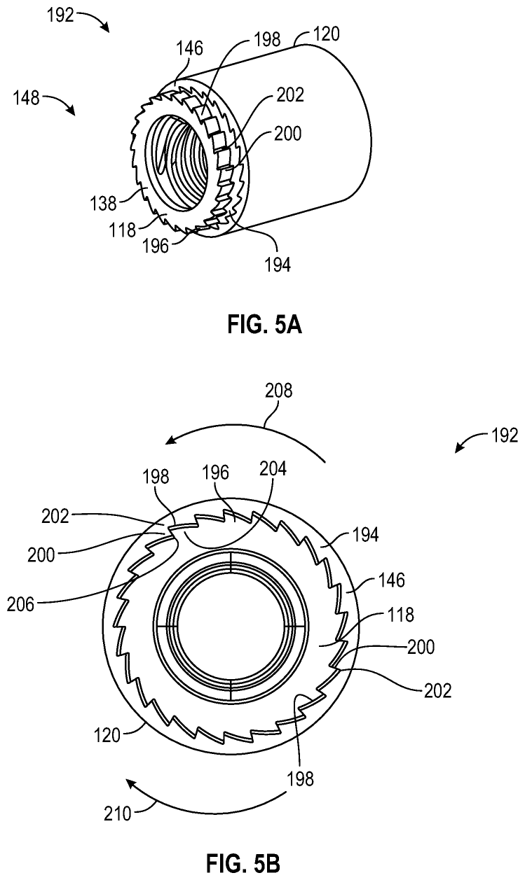

FIGS. 5A and 5B show perspective and end views, respectively, of an alternate embodiment of a multi-layer device 192 for disinfecting a needleless connector 106. In this embodiment, a modified ratchet and pawl mechanism replaces the snap elements 182, 184, 186, 188. The outer housing 120 includes a plurality of inner gear teeth 194 that extend from the chamber wall 146 at the chamber open end 148. The inner teeth 194 engage a plurality of outer gear teeth 196 located on the flange 138 of the inner housing 118. Each inner tooth 194 includes a gear tooth profile having a first slope 198 on a first face and a second slope 200 on a second face that is significantly steeper than the first slope 198. Each outer tooth 196 includes a first slope 204, first face, second slope 206 and second face that are substantially similar to the first slope 198, first face, second slope 200 and second face of each inner tooth 194 to form corresponding tooth profiles for the inner 194 and outer 196 teeth. In one or more embodiments, the second slopes 200, 206 may be substantially vertical to form a step 202.

When the inner 194 and outer 196 teeth are engaged, the outer housing 120 is rotatable in a first direction relative to the inner housing 118 but not rotatable in a second direction opposite the first direction. In particular, the outer housing 120 is rotatable with respect to the inner housing 118 when the first slope 198 of each inner tooth 194 slides over the first slope 204 of an outer tooth 196 (see arrow 208). Contact between the second slope 200 of each inner tooth 194 and second slope 206 of each outer tooth 196 prevents rotation of the outer housing 120 in an opposite direction (see arrow 210). Thus, the inner teeth 194 and outer teeth 196 serve as a ratchet and pawl arrangement, respectively.

As previously described, the outer housing 120 is rotated to provide frictional engagement between the engagement surface 168 and the access surface 112 to actively disinfect the access surface 112. Upon disinfection and at the end of outer housing 120 movement, the inner teeth 194 of the outer housing 120 engage with the outer teeth 196 of the inner housing 118. This causes a click sound, such as that which occurs in a known ratchet and pawl mechanism, due to contact and movement that occurs when the first slope 198 of each inner tooth 194 slides over the first slope 204 of an outer tooth 196. In accordance with the present disclosure, the click sound provides positive audible indication to a user such as a clinician, nurse or other health care provider that disinfection of the needleless connector 106 is complete. Further, engagement of the inner 194 and outer 196 teeth assists in the removal of the device 192 from the needleless connector 106 due to the outer housing 120 not being rotatable in the second direction.

The devices 100, 192 can be made from any of a number of types of plastic materials such as polycarbonate, polypropylene, polyethylene, glycol-modified polyethylene terephthalate, acrylonitrile butadiene styrene or any other moldable plastic material used in medical devices. In one or more embodiments, the device comprises a polypropylene, polyethylene material or polyurethane. In addition, the devices may be approximately 17 mm in diameter and 17 mm in length.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A disinfection device for disinfecting a female medical connector having first external threads and an access surface, the disinfection device comprising:
an inner housing including an inner wall that defines a hole that extends through the inner housing;
first internal threads formed on the inner wall that threadably engage the first external threads;
second external threads formed on a housing outer surface of the inner housing;
a flange formed on a flange end of the inner housing;
an outer housing having a chamber defined by a chamber wall, wherein the chamber forms a chamber open end having an end surface;
an absorbent material that extends through the chamber and hole and contacts the access surface wherein the absorbent material includes a disinfectant or antimicrobial agent;
second internal threads formed on the chamber wall that engage the second external threads to enable rotation of the outer housing and absorbent material to provide frictional engagement between the absorbent material and the access surface wherein the second internal and second external threads are configured to provide sufficient scrubbing time and scrubbing rotations by the absorbent material to disinfect the access surface and wherein the outer housing moves toward the inner housing due to rotation of the outer housing; and
at least one resilient snap element located on the end surface, wherein contact between the snap element and flange during movement of the outer housing toward the inner housing causes the snap element to deflect from an extended position wherein the snap element returns to the extended position when the flange clears the snap element wherein positioning of the snap element in the extended position after being deflected indicates that disinfection of the access surface is complete.

2. The disinfection device of claim 1, wherein a thread pitch of the second external threads is selected to provide sufficient scrubbing time and scrubbing rotations to actively disinfect the access surface.

3. The disinfection device of claim 1, wherein the second external threads are configured as fine threads as defined by the Unified Thread Standard.

4. The disinfection device of claim 1, wherein the second external threads include a breakable fin element that stops rotation of the inner housing relative to the outer housing to facilitate initial thread engagement between the first internal threads and first external threads when attaching the female connector to the inner housing.

5. The disinfection device of claim 1, wherein the snap element provides audible and visual indication that disinfection is complete.

6. The disinfection device of claim 1, wherein the snap element contacts the flange to hold the inner housing within the outer housing to enable removal of the female medical connector from the disinfection device by disengaging the first external threads of the female medical connector from the first internal threads of the inner housing.

7. The disinfection device of claim 1, wherein the absorbent material is attached to a mounting element extending from a closed end of the outer housing.

8. The disinfection device of claim 1, wherein the female medical connector is selected from a group consisting essentially of needleless connectors, catheter luer connectors, stopcocks, or hemodialysis connectors.

9. The disinfection device of claim 1, wherein the female medical connector is a needleless connector having an access surface, septum, hub surface and first external threads.

10. The disinfection device of claim 1, wherein the disinfectant or antimicrobial agent is selected from a group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorhexidine, chlorhexidine diacetate, chlorhexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof.

11. The disinfection device of claim 10, wherein the disinfectant or antimicrobial agent comprises at least one of chlorhexidine gluconate and chlorhexidine diacetate.

12. The disinfection device of claim 1, wherein the disinfectant or antimicrobial agent is a fluid or a gel.

13. A disinfection device for disinfecting a female medical connector having first external threads and an access surface, the disinfection device comprising:
an inner housing having an inner wall that defines a hole that extends through the inner housing;
first internal threads formed on the inner wall that threadably engage the first external threads;
second external threads formed on a housing outer surface of the inner housing;
a flange formed on a flange end of the inner housing, the flange including a flange outer surface;
an outer housing having a closed end and a chamber defined by a chamber wall, wherein the chamber forms a chamber open end having an end surface;
a mounting element extending from the closed end;
absorbent material attached to the mounting element, wherein the absorbent material extends through the chamber and hole and contacts the access surface wherein the absorbent material includes a disinfectant or antimicrobial agent;
second internal threads formed on the chamber wall that engage the second external threads to enable rotation of the outer housing and absorbent material to provide frictional engagement between the absorbent material and the access surface wherein the second internal and second external threads are configured to provide sufficient scrubbing time and scrubbing rotations by the absorbent material to actively disinfect the access surface and wherein the outer housing moves toward the inner housing due to rotation of the outer housing to compress the absorbent material and release the disinfectant or antimicrobial agent such that the disinfectant or antimicrobial agent contacts the hub portion to passively disinfect the hub portion; and
at least one resilient snap element located on the end surface, wherein contact between the snap element and flange during movement of the outer housing toward the inner housing causes the snap element to deflect from an extended position wherein the snap element returns to the extended position when the flange clears the snap element wherein positioning of the snap element in the extended position after being deflected indicates that disinfection of the access surface is complete and wherein the snap element contacts the flange outer surface to hold the inner housing within the outer housing.

14. The disinfection device of claim 13, wherein a thread pitch of the second external threads is selected to provide sufficient scrubbing time and scrubbing rotations to actively disinfect the access surface.

15. The disinfection device of claim 13, wherein the second external threads are configured as fine threads as defined by the Unified Thread Standard.

16. The disinfection device of claim 13, wherein the second external threads include a breakable fin element that stops rotation of the inner housing relative to the outer housing to facilitate initial thread engagement between the first internal threads and first external threads when attaching the female connector to the inner housing.

17. The disinfection device of claim 13, wherein the snap element provides audible and visual indication that disinfection is complete.

18. The disinfection device of claim 13, wherein the female medical connector is selected from a group consisting essentially of needleless connectors, catheter luer connectors, stopcocks, or hemodialysis connectors.

19. The disinfection device of claim 1, wherein the female medical connector is a needleless connector having an access surface, septum, hub surface and first external threads.

20. A disinfection device for disinfecting a female medical connector having first external threads and an access surface, the disinfection device comprising:
an inner housing having an inner wall that defines a hole that extends through the inner housing;
first internal threads formed on the inner wall that threadably engage the first external threads;
second external threads formed on a housing outer surface of the inner housing;
a flange formed on a flange end of the inner housing, the flange including a flange outer surface having a plurality of outer gear teeth each having a first slope and a second slope steeper than the first slope;
an outer housing having a closed end and a chamber defined by a chamber wall, wherein the chamber forms a chamber open end having an end surface;
a plurality of inner gear teeth located at the chamber open end that extend from the chamber wall wherein each tooth includes first and second slopes that correspond in profile to the first and second slopes of the outer teeth;
a mounting element extending from the closed end;
absorbent material attached to the mounting element, wherein the absorbent material extends through the chamber and hole and contacts the access surface wherein the absorbent material includes a disinfectant or antimicrobial agent; and
second internal threads formed on the chamber wall that engage the second external threads to enable rotation of the outer housing and absorbent material to provide frictional engagement between the absorbent material and the access surface wherein the second internal and second external threads are configured to provide sufficient scrubbing time and scrubbing rotations by the absorbent material to actively disinfect the access surface and wherein the outer housing moves toward the inner housing due to rotation of the outer housing to compress the absorbent material and release the disinfectant or antimicrobial agent such that the disinfectant or antimicrobial agent contacts the hub portion to passively disinfect the hub portion wherein,
upon disinfection of the access surface the inner teeth engage the outer teeth to cause a click sound due to movement between respective slopes of the inner and outer teeth wherein the click sound provides audible indication that disinfection of the access surface is complete.
